(19) United States
(12) Patent Application Publication
(10) Pub. No.: US 2022/0176147 A1
(43) Pub. Date: Jun. 9, 2022

(54) Wearable Micro-LED Healing Bandage

(71) Applicant: Marine Biology and Environmental Technologies, LLC, Tarzana, CA (US)

(72) Inventor: Eric A. Lewis, Tarzana, CA (US)

(73) Assignee: Marine Biology and Environmental Technologies, LLC, Tarzana, CA (US)

(21) Appl. No.: 17/677,945

(22) Filed: Feb. 22, 2022

Related U.S. Application Data
(63) Continuation of application No. 16/572,542, filed on Sep. 16, 2019.
Application No. 16/572,542 is a continuation of application No. 15/687,348, filed on Aug. 25, 2017, now abandoned.
(60) Provisional application No. 62/379,854, filed on Aug. 26, 2016.

(51) Int. Cl. A61N 5/06; A61L 15/00; A61F 13/00; A61M 1/00; A61F 13/02

(52) U.S. Cl. CPC A61N 5/0613; A61N 2005/0666; A61F 13/00063; A61M 1/90; A61F 13/00068; A61F 13/0206; A61F 13/0216; A61M 2205/051; A61M 2205/052; A61N 2005/0626; A61N 2005/0645; A61N 2005/0652; A61N 2005/0659; A61N 2005/066; A61L 15/00

(57) ABSTRACT

A wound dressing and method of use is provided that increases healing of tissues by targeting damaged tissue at a predetermined wavelength. The device includes the use of a negative pressure bandage, a flexible light sheet, and one or more bioactive marine extracts. Light emitted from the flexible light sheet penetrates through the bandage to target damaged tissue, which accelerates the wound healing process and works synergistically with the negative pressure bandage and bioactive marine extracts such as collagen fibers, alginate, chitosan and fucoidan, or any combination thereof to accelerate healing.

### CROSS-REFERENCE TO RELATED APPLICATIONS

This application claims benefit of U.S. Provisional Patent Application No. 62/379,854, filed Aug. 26, 2016, entitled “Wearable Micro-LED Healing Fabric Coupled with an Active Ointment,” the content of which is incorporated by reference in its entirety.

### FIELD OF THE DISCLOSURE

The present invention relates to a healing device and method, and more particularly, to the use of a wearable bandage having light emitting diodes (LEDs) and marine extracts to accelerate tissue healing.

### BACKGROUND OF THE INVENTION

The use of light for healing has long been practiced. Specific wavelengths of light have been used to treat various ailments and to stimulate the body's natural tissue healing abilities. LEDs that emit light in the red, far-red and infrared wavelengths have been shown to decrease pain, aid in wound healing, and increase skin rejuvenation. The use of this light for treating damaged tissue is known as “Red Light Therapy.” Although skin is naturally exposed to light, increasing exposure of wounded tissue to light in the far-red, red, and near infrared wavelengths accelerates wound repair.

The extent to which light interacts with biological tissues depends on characteristics and parameters of light devices, such as wavelength and dose, and also on the optical properties of the tissue. If applied properly, light therapy increases cellular proliferation, stimulates angiogenesis, increases local blood flow into existing and newly-formed capillaries allowing for more nutrients to circulate into the proliferating area, leading accelerated healing by a factor of two.

The mechanism of how red light therapy aids in tissue repair is not completely understood. However, despite the exact mechanism being unknown, numerous studies have shown its beneficial use. Red light has been shown to increase release of ATP, an important source of energy, which thereby aids in activity performance. (See “Light-emitting diode therapy in exercise-trained mice increases muscle performance, Cytochrome C oxidase activity, ATP and cell proliferation,” Ferraesi et al., J Biophotonics. 2016 September;9(9):976). Light, especially light in the red and near-infrared range, has been shown to have the ability to penetrate skin layers and energize fibroblast cells to produce collagen and elastin, which helps repair skin damage. LED light therapy has also been shown to increase growth of epithelial cells, improve recovery of musculoskeletal training injuries, and reduce pain in children suffering from oral mucositis. (See “Effect of Light-Emitting Diode Irradiation on Wound Healing,” Whelan H T, J Clin Laser Med Surg. 2001 December; 19(6):305-314). Animal studies have shown that LED light therapy increases cell growth in mouse-derived fibroblasts, rat-derived osteoblasts, rat-derived skeletal muscle cells and also has the ability to decrease wound size. Id.

One theory of mechanism is that red light is absorbed by the mitochondria. Metabolism in the mitochondria is usually restricted by a biologically active molecule called nitric oxide, which binds to cytochrome oxidase and prevents the mitochondria from using oxygen in the electron transport chain. Red light absorbed by the mitochondrial chromophores act by photodissociating the nitric oxide molecule from cytochrome C, which leads to increased ATP production, blood flow and nitric oxide release. Consequently, by exposing wounded tissue to red light, there is an increased secretion of growth factors, activation of enzymes and other secondary messengers to aid in tissue repair acceleration.

Many devices that use LEDs for healing use a matrix of LEDs on a matrix board, such as disclosed in European Pat. App. Pub. No. EP2044973A1 to Vibor. These types of LED devices are similar to tanning beds, except that instead of UV lamps in the device, the lamps are replaced with red LEDs. While these types of device may be effective at treating damaged tissue, they are bulky and expensive. They also require the user to dedicate several hours per week in order to receive a sufficient amount of light to observe positive effects. Another limitation of these types of devices is the devices cannot control with accuracy the distance of the light source to the target tissue since each patient within the bed is differently sized. Because light intensity varies with distance, cellular stimulation cannot be controlled with accuracy using these types of machines. Compounding this problem is that not only will low light intensity not stimulate cellular proliferation, but increased light intensity also has a negative effect on cellular stimulation. Thus, accurate control of light intensity on specific tissues is highly desirable.

Other types of devices have been used to aid in tissue repair. For example, light therapy devices have been used to treat topical wounds by placing a flexible matrix of LEDs over wounded tissue, such as disclosed in U.S. Patent Pub. No. 20070233208A1 to Kurtz et al. Other devices include LED handheld devices disclosed in U.S. Patent Pub. No. 20090088824A1 to Baird et al.

Use of red light therapy is not the only method to accelerate wound healing. Bioactive marine extracts (BAME) are known to accelerate the wound healing process. BAME increase the production of fibrocytes, which leads to increased production of collagen, elastin, hyaluronic acid and other compositions involved in would repair. Bioactive marine extracts can be derived from a wide variety of sources including but not limited to algae, shellfish and mollusks. Sources of algae bioactive marine extracts include: Macrocystis integrifolia, Ascophyllum nodosum, Fucus vesiculosus, and Spirulina pacifica. Sources of shellfish bioactive marine extracts include: Arthrospira platensis, Microsystis aeruginosa, Haliotis refescens, and Haliotis fulgens. Sources of mollusk bioactive marine extracts include: Holothuria Mexicana, Stichopus chlorontus, Crassostrea gigas, and Chlamys Rubida. Marine extracts used in tissue repair are disclosed U.S Patent Application Pub. No. 2014/0106001A1, entitled “Marine extract compositions and methods of use,” and U.S. Patent Application Pub. No. 2016/0228352, entitled “Marine extract compositions and methods of use,” to Lewis. Ointments, oils, serums, and lotions that use bioactive marine extracts are commercially available. Alone, bioactive marine extracts have been shown to increase wound healing time by a factor of three or four.

Although the exact mechanism of action of BAME healing effects is unknown, it is theorized that the extracts alter gene expression via several different signal transduction pathways. Bioactive marine extracts have been included in wound dressings that provide an optimal micro-climate through biologically active and biocompatible molecular arrangements. The dressings using BAME can deliver small-molecule modulators that interact with cell membrane proteins to cause a signal transduction that increase gene expression of proteins required in tissue repair.

Negative pressure therapy yet is another technology used to aid in wound repair. Negative pressure bandages, also called vacuum bandages, remove wound fluids by applying negative pressure suction to the wound area. Such pressure promotes healing by facilitating the granulated tissue at the wound site while simultaneously managing the fluid away from the tissue by using an absorbent material. By managing fluid away from the wound, swelling is reduced and increases blood flow. Various types of negative pressure devices have been developed to remove exudates, protect the wound and increase healing time. Examples of these devices are disclosed in U.S. Pat. No. 9,421,132 to Dunn, U.S. Pat. No. 8,992,492 to Anderson, U.S. Pat. No. 8,439,894 to Miller, U.S. Pat. No. 8,162,909 to Blott, U.S. Pat. No. 9,180231 to Greener, and U.S. Patent App. Pub. No. 2014/0343520 to Bennett. However, there still remains a need to improve and accelerate healing time for wounds and damaged tissue. All patents, patent applications and publications cited in this application are incorporated by reference, for all purposes, in their entireties.

### BRIEF SUMMARY OF THE PRESENT INVENTION

The present invention provides for devices that combine bio-active marine extracts (BAME) with light therapy to aid in the healing of wounds and decrease pain associated with wounds. The devices may include incorporation into bandages, masks, gloves, and full body suits. By providing a light delivery system as a wearable device, the device controls, with high accuracy, the light intensity reaching target tissues. The devices and associated methods accelerate wound healing by using light therapy, and in preferred embodiments use light in at least the red and near infrared spectrum. The addition of light sources emitting light in the blue or ultraviolet spectrums adds an antipathogenic feature to the device to further increase wound healing. Attaching a light therapy device that uses BAME to a vacuum bandage further increases the speed of wound healing compared to vacuum therapy or red light therapy alone. The use of BAME and red light therapy synergistically accelerates wound repair because BAME and red light repair tissue through distinct mechanisms. BAME enhances the stem cells of target tissues, creating new fibrocytes and rejuvenating existing fibrocytes, while red light therapy increases capillary growth, leading to increased blood flow, and delivering nutrients to the generative tissue tissues. Both induce DNA expression level changes but through different means. BAME targets cells through chemical reactions with specific receptors located in the cell membranes, and far and deep red light induces DNA expression changes in target cells via electromagnetic waves associated with the light sources that can pass through the membranes and generate quantum cascades in the intracellular medium.

In one embodiment of the invention, there is a wound dressing for delivering light energy to treat damaged tissues. The wound dressing includes a flexible light sheet having a plurality of light sources capable of providing at least one effective wavelength at an effective intensity to stimulate cell proliferation on a target area of a patient. The wound dressing also includes a light source controller operatively connected to the flexible light sheet to control the plurality of light sources. The wound dressing further includes a translucent bandage connected to the flexible light sheet, the translucent bandage capable of permitting light energy emitted from the plurality of light sources to pass through the translucent wound dressing to the target area of the patient. The translucent bandage includes an absorbent material to draw exudate from the wound. The wound dressing also includes a therapeutic medicament layer adjacent to the translucent bandage, the therapeutic medicament layer has at least one medicament selected from the group consisting of at least one of collagen fibers, alginate, chitosan, and fucoidan.

In another embodiment of the wound dressing, the translucent bandage is characterized as a negative pressure bandage that has a vacuum reservoir positioned between the flexible light sheet and the therapeutic medicament layer, as well as includes a vacuum pump, and a power source connected to the vacuum pump.

In still another embodiment, there is method of treating wounds and includes the steps of placing the wound dressing on a target area of a patient, and illuminating the target area of the patent with the plurality of light sources for an effective amount of time and an effective intensity sufficient to cause cell proliferation at the target area of the patient.

### BRIEF DESCRIPTION OF THE DRAWINGS

FIG. 1 is a perspective view of a wound dressing having a negative pressure bandage and flexible light sheet;

FIG. 2 is an exploded view of a negative pressure bandage without the flexible light sheet;

FIG. 3 is a perspective view of a flexible light sheet, conductive pathway and enclosing film;

FIG. 4 is a cross sectional view of an optical guide for directing light toward a treatment area;

FIG. 5 is a cross sectional view of a wound treatment area covered by a therapeutic medicament layer, negative pressure bandage, and flexible light sheet without negative pressure applied to the bandage;

FIG. 6 is a cross sectional view of a wound treatment area covered by a therapeutic medicament layer, negative pressure bandage, and flexible light sheet that conforms to the shape of the wound when negative pressure is applied to the bandage.

### DETAILED DESCRIPTION OF EMBODIMENTS

The invention now will be described more fully hereinafter with reference to the accompanying drawings, in which embodiments of the invention are shown. This invention may however be embodied in many different forms and should not be construed as limited to the embodiments set forth herein. Rather, these embodiments are provided so that this disclosure will be thorough and complete, and will fully convey the scope of the invention to those skilled in the art.

It will be understood that when an element is referred to as being “on” another element, it can be directly on the other element or intervening elements may be present therebetween. As used herein, the term “and/or” includes any and all combinations of one or more of the associated listed items.

It will be understood that, although the terms first, second, third etc. may be used herein to describe various elements, components, regions, layers, and/or sections, these elements, components, regions, layers, and/or sections should not be limited by these terms. These terms are only used to distinguish one element, component, region, layer, and/or section from another element, component, region, layer, and/or section.

It will be understood that the elements, components, regions, layers and sections depicted in the figures are not necessarily drawn to scale.

The terminology used herein is for the purpose of describing particular embodiments only, and is not intended to be limiting of the invention. As used herein, the singular forms “a,” “an,” and “the” are intended to include the plural forms as well, unless the context clearly indicates otherwise. It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

Furthermore, relative terms, such as “lower” or “bottom,” “upper” or “top,” “left” or “right,” “above” or “below,” “front” or “rear,” may be used herein to describe one element's relationship to another element as illustrated in the Figures. It will be understood that relative terms are intended to encompass different orientations of the device in addition to the orientation depicted in the Figures.

Unless otherwise defined, all terms used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

Exemplary embodiments of the present invention are described herein with reference to idealized embodiments of the present invention. As such, variations from the shapes of the illustrations as a result, for example, of manufacturing techniques and/or tolerances, are to be expected. Thus, embodiments of the present invention should not be construed as limited to the particular shapes of regions illustrated herein but are to include deviations in shapes that result, for example, from manufacturing. The invention illustratively disclosed herein suitably may be practiced in the absence of any elements that are not specifically disclosed herein.

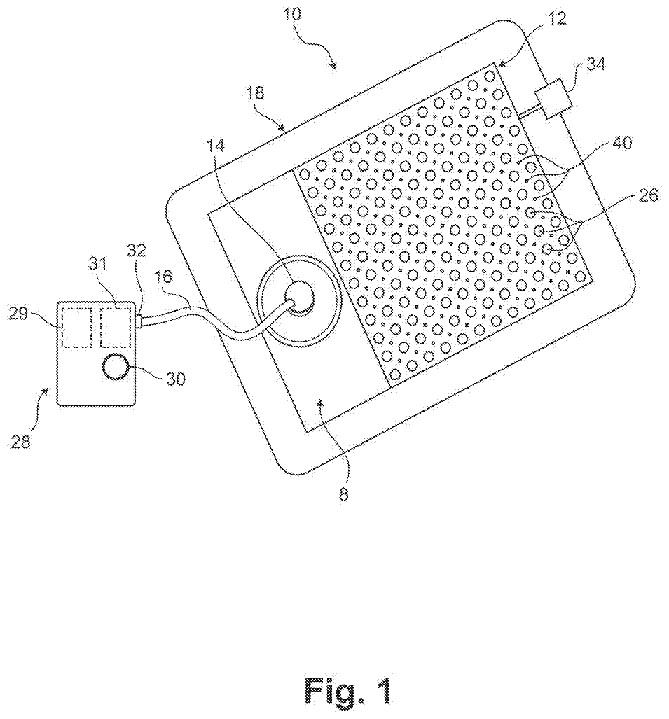

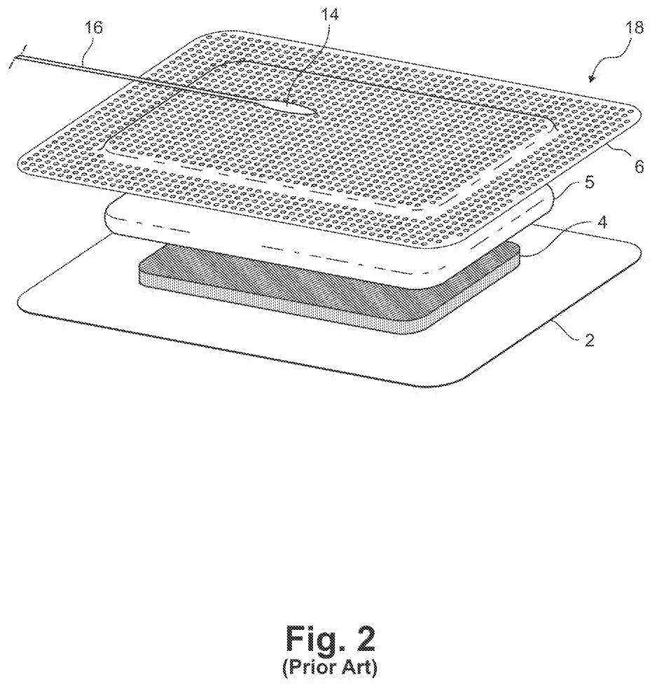

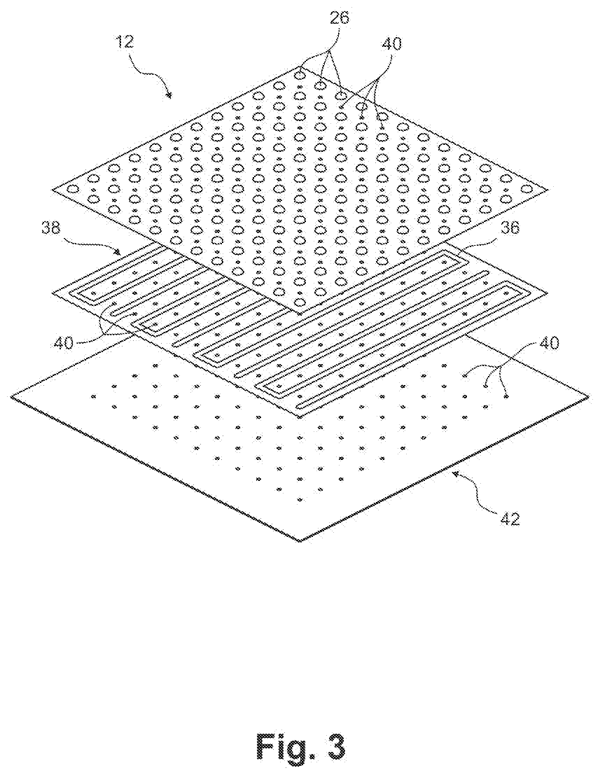

Referring now to FIGS. 1-3, FIG. 1 is a perspective view of an embodiment of a wound dressing 10 having a negative pressure bandage 18, power and vacuum unit 28 and flexible light sheet 12. FIG. 2 illustrates the negative pressure bandage 18 without the power and vacuum unit 28 or flexible light sheet 12. FIG. 3 illustrates components of the flexible light sheet 12 and surrounding layers.

The wound dressing 10 promotes healing via a combination of a negative pressure bandage 18, flexible light sheet 12 and therapeutic medicament layer 20. Numerous types of negative pressure bandages 18 have previously been described and may be incorporated into the present invention. FIGS. 1 and 2 illustrate one such bandage that can be used in the present invention.

The bandage 18 includes an adhesive contact layer 2, which can be a silicone adhesive layer that protects the wound environment by preventing particulates from entering the wound 22. On top of the contact layer 2 is a vacuum reservoir 4. The vacuum reservoir 4 can be an enclosed pocket or an absorbent material that acts as a vacuum reservoir and absorbs exudate in an airlock layer. The present embodiment illustrates the vacuum reservoir as an airlock layer that enables distribution of negative pressure across the wound 22 while enabling movement of exudate through the dressing. Above the vacuum reservoir 4 is an absorbent core 8 that locks exudate away from the wound 22. Above the absorbent core 8, and in contact with the adhesive layer 2 is a top film layer 6. Preferable, the top film layer 6 has a high moisture vapor transmission rate to transpire exudate. The top film layer 6 includes a vacuum port 14 so that a vacuum can be applied to the negative pressure bandage 18. A flexible tubing 16 extends from the vacuum port 14 to a tubing connector 32 located on a power and vacuum unit 28. The power and vacuum unit 28 can be separate structures but in a preferred embodiment the power source 29 (such as batteries) and vacuum source and pump 31 are enclosed within the single power and vacuum unit 28 to provide easy portability of the device. The power and vacuum unit 28 includes a power button 30 to effectuate a vacuum within the negative pressure bandage 18. The suction capacity of the negative pressure bandage 18 should be sufficient such that the wound is in contact with bandage 18 or layers beneath the bandage 18 such as the adhesive contact layer 2. A negative pressure of −80 mmHg65 is sufficient for treating wounds but those having ordinary skill in the art will appreciate that lower and higher negative pressures can be used to conform the bandage 18 to the treatment area and remove exudate. The bandage 18 should allow optical scattering so that the target treatment area is exposed to emitted light. The bandage 18 should be a translucent bandage that has properties that permit at least some light to pass from LEDs 26 to the target area on the patient. In other embodiments, the bandage 18 may not be a negative pressure bandage but can be a traditional non-negative pressure bandage.

One commercial negative pressure bandage that can be used in the present invention is the PICO Single Use Negative Pressure Wound Therapy (NPWT) from Smith & Nephew. Other types of negative pressure bandages are disclosed in U.S. Pat. No. 8,956,336 to Haggstrom et al., U.S. Pat. No. 9,381,283 to Adams et al., and U.S. Pat. No. 8,350,116 to Lockwood et al., the contents of each are incorporated by reference in their entireties.

Turning specifically the flexible light sheet 12 shown in FIG. 1 and FIG. 3, the flexible light sheet 12 is positioned above the top film layer 6 of the negative pressure bandage 18 and may be attached to the top film layer 6 or other layer of the wound dressing 10 by an adhesive. However, those having ordinary skill in the art can appreciate that a flexible sheet 12 can be positioned in other regions of the wound dressing 10 without departing from the spirit of the invention as long as the light emitted from flexible sheet 12 can penetrate the treatment area of a patient. The flexible light sheet 12 has a plurality of light sources 26. In a preferred embodiment, these light sources 26 are LEDs that can emit light in the red, far-red, and near infrared spectrum. Light in this range is known to accelerate the healing process of wounded tissues. Numerous types of light sheets may be used to provide sufficient light to treat a wounded area. In the embodiment shown in FIG. 3, the light sheet 12 is made of a porous silicon film of about 50 microns thick, which encloses an array of 144 wide angle red light LEDs of 10 microns that can emit light having wavelengths ranging from 580 to 700 nm. In other embodiments, the array comprises at least 50 LEDs. Adjacent to the flexible light sheet 12 is a conductive pathway film 38. This film 38 includes a conductive pathway 36 that provides current to illuminate the LEDs 26. In one embodiment, the conductive pathway 36 is a copper pathway of about 10 microns thick engraved on a porous silicon film of about 50 microns thick. The flexible light sheet 12 may comprise a reflective backing layer to prevent light from the plurality of light sources from diffusing away from the treatment area.

Light therapy bandages and flexible sheets having embedded emitters for phototherapy are disclosed in United States Patent Application Pub. No. 2007/0233208 to Kurtz et al., and U.S. Pat. No. 6,290,713 to Russell et al. and are incorporated by reference in their entireties. Numerous types of LED light arrays may be used in the present invention, such as the SST-10-660-B90 LED manufactured by Luminus Devices, Inc., and should be configured so that the optical scatter allows a light flux of about than 50 mW/cm² to the target area when powered by a current of I₀<150 mA. Those in the art will appreciate that other LEDs will also work to provide sufficient light to a wound treatment site and may provide light flux between 40 mW/cm² and 60 mW/cm², preferably around 50 mW/cm². The flexible light sheet 12 is operatively connected to a light source controller 34 than can power the LEDs 26 and is capable of controlling one or more of the wavelength of light emitted, time of emission, and intensity of light.

Adjacent to the conductive pathway film 38 is a light array enclosing film 42. In one embodiment, the light array enclosing film 42 is a silicon film of about 50 microns thick that seals the flexible light sheet 12 and conductive pathway film 38. The flexible light sheet 12, conductive pathway film 38, and light array enclosing film 42 may have pores 40 that allow exudate to evaporate to aid in the wound healing process by removing excess fluid from the wound area 22.

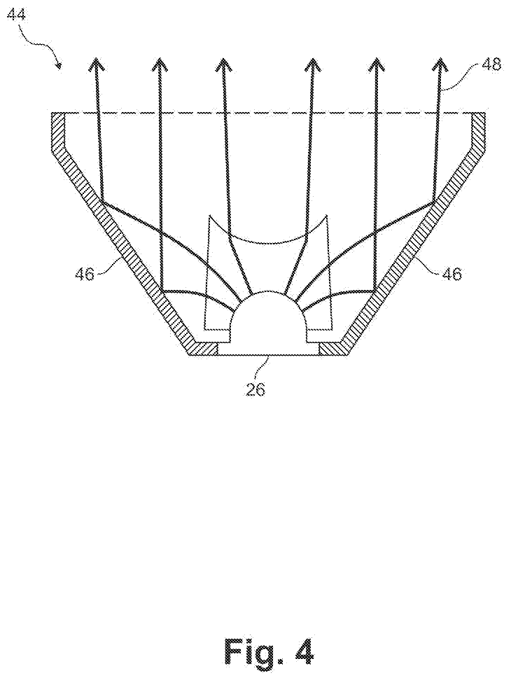

To better direct light toward the treatment area, the LEDs 26 may each be surrounded by an optical guide 44, as shown in FIG. 4. The optical guide 44 prevents light 48 from the LED 26 from diffusing in a direction away from the treatment area, and directs light perpendicular to the flexible light sheet 12. The optical guides 44 have angular side walls 46 such that when the light is emitted from the LED in all directions, the angular side walls reflect light in a generally perpendicular manner so that the treatment area receives maximum exposure to the light.

In addition to the LEDs 26 on the flexible light sheet 12 being capable of emitting red light, LEDs may be incorporated that are capable of emitting light in the blue or UV wavelength. Light in the blue and UV wavelength can kill pathogens and help sterilize the wound area before treating the wound area with red light. In one embodiment, the wavelength of blue light is between 420 nm and 490 nm, and preferably about 476 nm. Deep red wavelengths that are able to penetrate the first layer of tissue, are between 660 nm and 700 nm, preferably around 670 nm, and can penetrate tissue to a depth of about 8-10 mm. Far-red wavelengths between 700 nm and 800 nm (preferable around 720) are able to penetrate tissue more than 10 mm, but generally less than 25 mm. Infrared wavelengths between 800 nm and 1400 nm (preferably around 880 nm) are able to penetrate tissues around 25 mm. The combination of using LEDs 26 having the capability of emitting light in the blue (to disinfect the wound), deep red, far-red and infrared allows three-dimensional healing of the wound 22. Those having skill in the art will appreciate that a single adjustable wavelength LED source may be capable of emitting light at these various wavelengths. In another embodiment, the wound dressing 10 can incorporate different single wavelength emission LEDs to cover the spectrum described above.

In another embodiment of the invention, the wound dressing 10 incorporates a therapeutic medicament layer 20. The medicament layer 20 may be used with or without the negative pressure bandage 18.

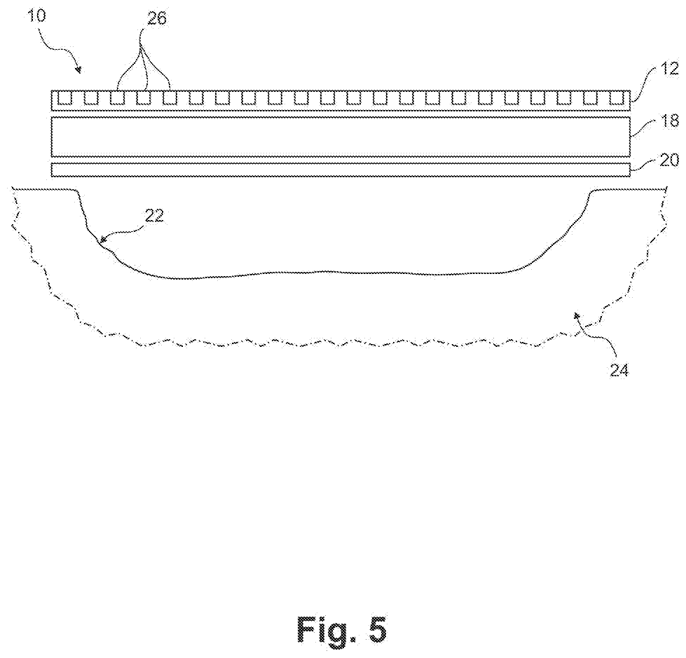

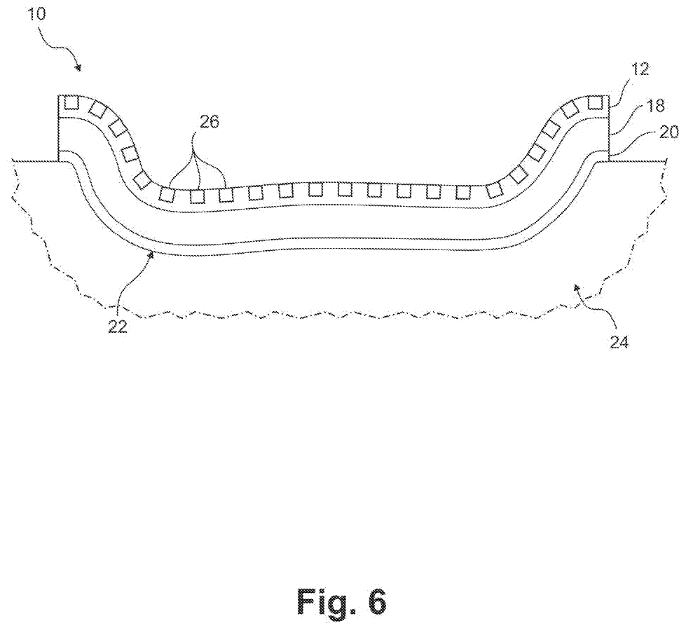

FIG. 5 and FIG. 6 illustrate cross sectional views of the wound dressing on top of the patient's tissue 24 having a wounded area 22. FIG. 5 shows the wound dressing 10 when there is no negative pressure (i.e. no vacuum) applied to the wound dressing 10 and FIG. 6 shows the wound dressing 10 when negative pressure is applied. When applied, the wound dressing 10 conforms to the shape of the wound 22, thus bringing the wound 22 in direct contact with the therapeutic medicament layer 20 and closer in proximity to the flexible light sheet 12.

The dressing 10 in FIGS. 5 and 6 include the flexible light sheet 12, negative pressure bandage 18 and therapeutic medicament layer 20. The therapeutic medicament layer 20 may be applied in a variety of embodiments. In one embodiment, the therapeutic medicament layer 20 is a gauze pad or other absorbent woven or non-woven material, such as a polyester non-woven fabric. A preferred embodiment is a 12-ply porous gauze pad having a thickness of about 1.5 mm. The concentration of bioactive marine extracts can be a variety of ranges from 0.1% to 50% and in a preferred embodiment ranges from 0.5% to 10%. In a more preferred embodiment the concertation of bioactive marine extracts is about 2% and the gauze pad also includes almond oil, Lipoderm or Versa cream. Preferably the gauze pad is retrieved from a one-time use sealed and sterilized package, such as the McKesson Medi-Pak™ sterile performance gauze sponges.

As shown in FIG. 5, the wound 22 craters below the medicament layer 20. However, when the medicament layer 20 is used in conjunction the wound dressing 10, and negative pressure is applied, the bandage 18 and medicament layer 20 conform to the shape of the wound, thus providing better contact with the wound 22, thereby improving healing time.

The therapeutic medicament layer 20 may include a variety of bioactive marine extracts, such at least one of collagen fibers, alginate, chitosan and fucoidan, or any combination thereof. Other bioactive marine extracts include algaes: Macrocystis integrifolia, Ascophyllum nodosm, Fucus vesiculosus, and Spirulina pacific. Bioactive marine extracts derived from shellfish that aid in wound healing include: Arthorospira platensis, Microsystis aeruginosa, Haliotis rufescens, and Haliotis fulgens. Bioactive marine extracts derived from mollusks for use in the medicament layer include Holothuria Mexicana, Stichopus chlorontus, Crassostrea gigas and Chlamys Rubida. Combinations of these disclosed bioactive marine extracts for use in tissue repair are disclosed in U.S. Patent Application Pub. Nos. 2014/0106001 and 2016/0228352 to Lewis, which are incorporated by reference in their entireties.

The frequency of the treatment and time of exposure impart the total amount of energy directed to the target area. In one embodiment, the irradiance is about 50 mW/cm² to the target area and the frequency of treatment and time of exposure allows for between 4 J/cm² per 12-hour period or 8 J/cm² per 24-hour period. A delivery of about 4 J/cm²+/−10% is preferred per 12 hour period, but should be at least 3 J/cm². Below that range generally produces minimal or no cellular proliferation benefit. Irradiance about 4.1 J/cm² begins to see a decrease in healing effects. Irradiance above 4.5 J/cm² shows negative effects for wound healing. Since dosage is defined as the product of the light power reaching the targeted tissue, multiplied by the time of exposure, the healing effectiveness of using red light is highly sensitive to the irradiance parameters. Interestingly, damaged tissue exposed energy much greater than 8 J/cm² in a 24-hour period would have diminishing effects and would lead to tissue healing a rate no greater than tissue being exposed to ambient light.

In other embodiments, the wound dressing 10 is operatively connected to a smart interface, such as a smart phone or computer that can interface with the flexible light sheet 12, such as the embodiments described in U.S. Pat. No. 9,370,449, entitled, “Phototherapy Dressing for Treating Psoriasis,” to Anderson et al., which describes processors configured by control logic to monitor and perform dose calculations and exposure time using smartphones, iPads, computers, and the like. The logic board of the smart device should be configured in such a way that the parameters of light wavelength, light intensity and duration of the light emission can be tuned and adapted to each user and each treatment. This way, the light emitted can be controlled to deliver the optimum results to a specific patient having a specific type of wound.

In other embodiments, the combination two or more of negative pressure bandages, medicament layers, and LED arrays can be incorporated into masks, gloves, body suits, gum devices, aural, vaginal, anal or nasal devices to accelerate wound healing.

Another embodiment of the invention includes a method of treating wounds by using the above described wound dressing 10. The method includes the steps of placing the wound dressing on the target area of a patient and illuminating the target area of the patient with the plurality of light sources for an effective amount of time and effective intensity sufficient to cause cell proliferation at the target area of the patient. The plurality of light emitting sources may emit light at a wavelength between 580 nm and 700 nm and the effective intensity has a flux of at least 50 mW/cm². The effective amount of time and effective intensity provide at least 4 J/cm² per 12-hour period to the target of a patient. In other embodiments, a negative pressure is applied to the wound dressing, thereby creating a suction force between the wound area and the target area of the patient, thereby causing the wound dressing to conform to the target area of the patient.

In another embodiment of the method of treating wounds, illuminating the target area is characterized as illuminating the target area with a blue light source, a deep red light source, far-red light source, and infrared light source. Illumination may be accomplished by using LEDs that are capable of tuning light wavelengths to a plurality of different wavelengths or by using single wavelength emission LEDS. In one embodiment, the blue light source has a wavelength between 420 nm and 490 nm, the deep red light source has a wavelength between 660 nm and 700 nm, the far-red light source has a wavelength between 700 nm and 800 nm, and the infrared light source has a wavelength between 800 nm and 1400 nm. The device should provide a dosage of between 40 mW/cm² and 60 mW/cm², preferably around 50 mW/cm². Light in each of the wavelengths can be provided simultaneously or sequentially in any order to provide the recommended dosage, equivalent to about 4 J/cm² to the target area for maximum healing effects. In some embodiments, only one light source wavelength is used during the illuminating step, while in other embodiments, two, three, or four of the above described wavelengths of light sources are used in the illuminating step.

The amount of time that each light wavelength should remain on understandably can vary with the intensity of the light since dosage is calculated as intensity of light multiplied by time of exposure. In one embodiment, the time should about 400 seconds for each light wavelength to provide the recommended 4 J/cm² to the target area. Ranges of illumination time between 1 minute and 20 minutes per 12-hour period may also provide sufficient results depending on the intensity of the light emitted.

In another embodiment of a method for treating wounds, stem cells from a patient are injected into the wound area before placing the wound dressing 10 on the patient. Stem cell injections to aid in wound healing is known in art and described in U.S. Pat. No. 8,119,398 to Sayre et al., incorporated by reference herein, which describes injecting adipose-derived stem cells for tissue regeneration and wound healing.

### LIST OF REFERENCE NUMERALS

2 Adhesive contact layer

4 Vacuum reservoir

6 Top film layer

8 Absorbent layer

10 Wound dressing

12 Flexible light sheet

14 Vacuum port

16 Flexible tubing

18 Negative pressure bandage

20 Medicament layer

22 Wound

24 Patient tissue

26 Light source/LEDs

28 Power and vacuum unit

29 Power source

30 Power switch

31 Vacuum source/pump

32 Tubing connector

34 Light source controller

36 Conductive pathway

38 Conductive pathway film

40 Pores

42 Film enclosing light array

44 Optical guide

46 Angular side wall

48 Light

What is claimed is:

1. A wound dressing for delivering light energy to treat medical conditions in damaged tissues, the wound dressing comprising:
a flexible light sheet having a plurality of light sources capable of providing at least one effective wavelength at an effective intensity to stimulate cell proliferation on a target area of a patient;
a light source controller operatively connected to the flexible light sheet to control the plurality of light sources;
a translucent bandage connected to the flexible light sheet, the translucent bandage capable of permitting light energy emitted from the plurality of light sources to pass through the translucent wound dressing to the target area of the patient, wherein the translucent bandage is comprised of an absorbent material; and
a therapeutic medicament layer adjacent to the translucent bandage, the therapeutic medicament layer comprising at least one medicament selected from the group consisting of at least one of collagen fibers, alginate, chitosan, and fucoidan.

2. The wound dressing of claim 1, wherein the plurality of light sources are a plurality of light emitting diodes (LEDs), and the effectively wavelength is a wavelength within the infrared or near infrared spectrum having wavelengths between 580 and 700 nm.

3. The wound dressing of claim 1, wherein the plurality of light sources are arranged in an array and the flexible light sheet comprises a porous silicon film.

4. The wound dressing of claim 1, wherein the flexible light sheet is a porous silicon film surrounded an array of at least 50 wide angle LEDs capable of emitting a wavelength between 580 nm and 700 nm.

5. The wound dressing of claim 1, wherein the flexible light sheet comprises a reflective backing layer, thereby preventing light from the plurality of light sources from diffusing away from the treatment area, and wherein each of the plurality of light sources is surrounded by an optical guide to direct light through the translucent bandage to the treatment area.

6. The wound dressing of claim 1, wherein the controller is operatively connected to the plurality of light sources via conductive elements embedded on or within the flexible light sheet to power the plurality of light sources.

7. The wound dressing of claim 1, wherein the therapeutic medicament layer comprises at least two of collagen fibers, alginate, chitosan, and fucoidan.

8. The wound dressing of claim 1, wherein the collagen fibers, alginate, chitosan and fucoidan are of a marine origin.

9. The wound dressing of claim 1, wherein fucoidan is a sulfated polyfucose polysaccharide.

10. The wound dressing of claim 1, wherein the sulfated polyfucose polysaccharide is derived from brown marine algae.

11. The wound dressing of claim 1, wherein the therapeutic medicament layer is comprised of at least one marine extract.

12. The wound dressing of claim 1, wherein the therapeutic medicament layer is a gauze layer.

13. The wound dressing of claim 1, wherein the translucent bandage is characterized as being a negative pressure bandage, the negative pressure bandage comprising a vacuum reservoir positioned between the flexible light sheet and the therapeutic medicament layer, a vacuum pump, and a power source connected to the vacuum pump.

14. The wound dressing of claim 13, wherein the light therapy bandage further comprises an absorbent layer, a top film layer on top of the absorbent layer, a vacuum port positioned on top of the top film layer, and adhesive contact layer below the absorbent layer, and a flexible tubing connecting the vacuum pump to the vacuum port.

15. A method for treating wounds, comprising the steps of:
a) placing the wound dressing of claim 1 on a target area of a patient; and
b) illuminating the target area of the patient with the plurality of light sources for an effective amount of time and an effective intensity sufficient to cause cell proliferation at the target area of the patient.

16. The method of claim 15,
wherein the plural of light sources emit light at a wavelength between 580 nm and 700 nm;
wherein the effective intensity has a flux of at least 50 mW/cm²; and,
wherein the effective amount of time and the effective intensity provide at least 4 J/cm² per 12-hour period to the target area of the patient.

17. The method of claim 15, further comprising the step of
applying a negative pressure to the wound dressing thereby creating a suction force between the wound dressing and the target area of the patient, thereby causing the wound dressing to conform to the target area of the patient.

18. The method of claim 15,
wherein illuminating the target area is characterized as illuminating the target area of a patient with the plurality of light sources, wherein the plurality of light sources includes:
i) a blue light source having a wavelength between 420 nm and 490 nm,
ii) a deep red light source having a wavelength between 660 nm and 700 nm,
iii) a far-red light source having a wavelength between 700 nm and 800 nm, and
iv) an infrared light source having a wavelength between 800 nm and 1400 nm,
wherein each of the blue light source, the deep red light source, the far-red light source, and the infrared light source each provide a dosage of between 40 mW/cm² and 60 mW/cm².

19. The method of claim 18, wherein illuminating the target area is performed sequentially with the blue light source, the deep red light source, the far-red light source, and the infrared light source, in any order.

20. The method of claim 16, further comprising the step of injecting stem cells from the patient in the target area of the patient.
